(19) United States
(12) Patent Application Publication
(10) Pub. No.: US 2007/0189975 A1
(43) Pub. Date: Aug. 16, 2007

(54) Hydrophilic matrix for delivery of active agent and product containing same

(76) Inventor: Timothy Thomson, West Newbury, MA (US)

Correspondence Address: KRAMER LEVIN NAFTALIS & FRANKEL LLP;INTELLECTUAL PROPERTY DEPARTMENT, 1177 AVENUE OF THE AMERICAS, NEW YORK, NY, 10036, US

(21) Appl. No.: 10/588,366

(22) PCT Filed: Feb. 2, 2005
(86) PCT No.: PCT/US05/03383

§ 371 Date: Aug. 2, 2006

Related U.S. Application Data
(60) Provisional application No. 60/541,099, filed on Feb. 2, 2004.
(60) Provisional application No. 60/544,618, filed on Feb. 13, 2004.

(51) Int. Cl. A61K 47/48

(52) U.S. Cl. 424/10.3; 424/486

(57) ABSTRACT

A sustained drug delivery system is disclosed comprising an open cell or reticulated, hydrophilic polyurethane.

### BACKGROUND OF THE INVENTION

There is a need for the delivery of pharmaceuticals, disinfectants and like to control or prevent disease or remediate unsanitary conditions. Such systems must deliver a calculated or measured amount of drug or disinfectant per unit time during use. If the method of delivery is by diffusion of an active ingredient out of a polymer matrix, Fick's Law predicts that the active ingredient will be delivered at the highest rate and each successive use will deliver active ingredient at a lower rate.

It has also been recognized that consumers will sometimes use a product which delivers an active ingredient over time beyond the point considered by the manufacturer to be the product's useful life. For example, active ingredients such as soaps are delivered from sponge materials but the soap is eventually depleted rendering the sponge ineffective. This is particularly problematic where the purpose of the device is to deliver a soap or disinfectant or the like e.g., where the active ingredient is important to maintain some critical sanitary condition. Where the purpose of the product is to deliver a disinfectant, the inability to tell when the disinfectant is exhausted leaves the consumer with the predicament of either buying a replacement product before it is necessary to do so, or more often of using a product after it has lost its effectiveness.

A system that either detects the continued delivery of an active ingredient or, alternatively, indicates the number of actual uses of a device would have great utility. This is especially the case where the ingredient functions to control disease, e.g. a disinfectant.

### DETAILED DESCRIPTION OF THE INVENTION

It has now been found that these problems can be solved by dispersing or dissolving an active ingredient in a soluble or erodible polymer matrix and dispersing the matrix onto an open cell or reticulated polymer scaffold. The soluble polymer matrix dispersed onto the scaffold can be used as a flow through or wipe on applicator. The rate of delivery of the active ingredient from the matrix is controlled by the rate of dissolution of the soluble polymer matrix as opposed to the diffusion of the active ingredient from that matrix. This achieves a desirable linear or “zero order” release of the ingredient.

The soluble or erodible polymer matrix containing the active ingredient can be dispersed in hydrophilic polyurethane. Alternatively, the soluble or erodible polymer matrix containing the active ingredient can be dispersed in a complex comprising hydrophilic polyurethane and hydrophobic polyurethane as taught in Thomson, U.S. Pat. No. 6,617,014. Such composites, methods for making them and the numerous end uses for the composites are described in U.S. Pat. No. 6,617,014 and in related U.S. Patent Publication US-2002-0018884-A1 and its PCT counterpart WO/01/74582 A1. The details of same are therefore known to the art. These details are specifically referred to and incorporated herein.

It has also been discovered that indicator compositions can be made according to the invention. Hydrophilic polyurethane is stained by many indicator molecules. If an active ingredient has a characteristic pH, the color of the indicator molecule responds to the pH of the active ingredient until the active ingredient is exhausted from the composition. Once the active ingredient is exhausted, the indicator will change color. Alternatively, if the indicator molecule responds to a particular chemical moiety, active ingredients containing that moiety may be similarly detected by the indicator.

The active ingredient is dissolved, absorbed or otherwise included within a soluble or erodible water soluble polymer. When the polymer erodes or dissolves, the active ingredient is released into the environment in which the product is being used and causes the indicator to retain its color.

The soluble or erodible polymer matrices contain broadly from 0.01 to 20.0% by weight of polymer of the substance to be released, desirably 0.05 to 10.0% by weight and preferably 0.1 to 5.0% by weight thereof. Any suitable polymer can be used as are known to those skilled in the art.

The substance to be released may be a soap, disinfectant, detergent or the like or may be a protein, sugar or other organic compound. The substance to be released may also be a fluorescent or spectrophotometric molecule or the like. The foregoing are examples but no limitation on the breadth of selection of either the soluble or erodible polymer or the substance to be released is implied.

Compositions containing hydrophilic polyurethane or composites containing a hydrophilic polyurethane foam in a hydrophilic substrate, as taught in U.S. Pat. No. 6,617,014, may contain broadly 0.01 to 25% polymer containing substance to be released, desirably 0.05 to 20% and preferable 0.10 to 10% polymer containing substance to be released by weight of the hydrophilic polyurethane or composite containing same.

The indicator molecules used in the compositions of the invention may be any molecule which through physical, physical-chemical or chemical interaction with active ingredient undergoes a discernible change in color, opacity or other characteristic and can thereby indicate the presence or absence of active ingredient in the environment in which the product is used.

### EXAMPLE 1

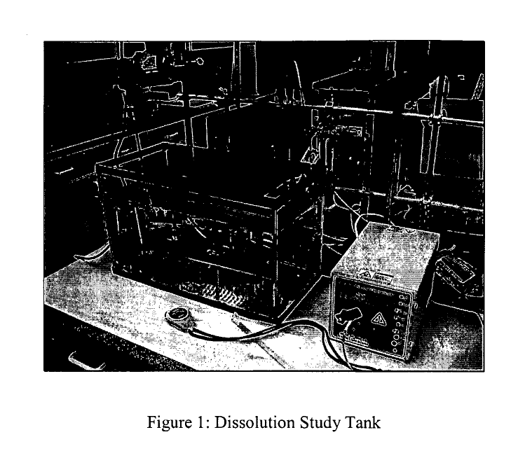

To study the dissolution rate from the composites of the invention a 30-liter aquarium as shown in FIG. 1 was used for the solution studies. It was fitted with a standard lab mixer. For each experiment the tanks were filled with 25 liters of tap water. Enough NaOH was added to make the water basic. The temperature was adjusted to 22° C. but not thereafter controlled. A sample of the water was used to set a visible spectrophotometer to 100% transmission.

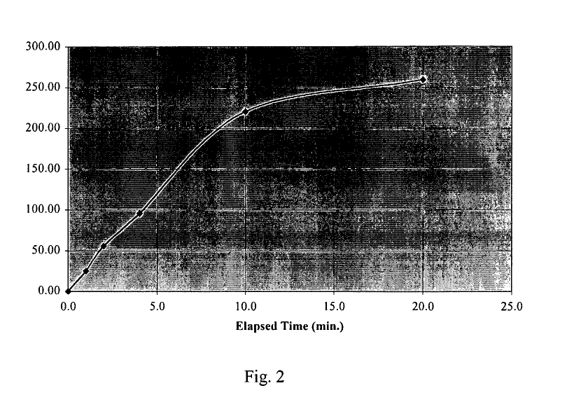

100 mg of bromothymol blue was imbibed into a piece of hydrophilic polyurethane foam, a common controlled release matrix.. After drying, the foam was placed in the test chamber and the rate of release of the dye was determined as described above. FIG. 2 shows the spectral data as a function of time. By the use of a calibration curve, these % transmission values at 617 nM can be translated into mass.

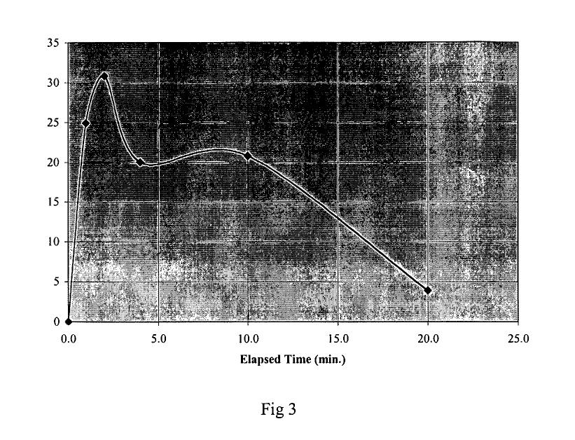

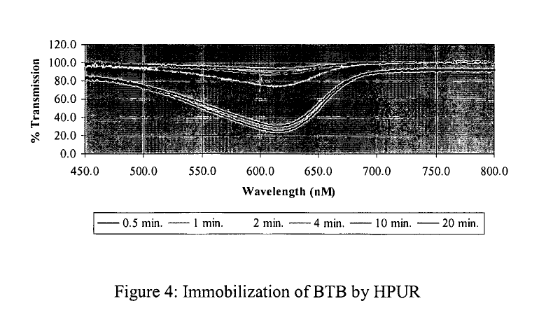

The rate of change of the delivery rate is seen in FIG. 3. FIG. 3 is a typical diffusion curve. The rate of release is a function of the concentration. EXAMPLE 2

Five grams of each of Pluronic F87, F108 and F127 were placed in an aluminum weighing pan. 100 mg of bromothymol blue (BTB) was weighed in to each. The pans were placed in an oven at 95° C. The Pluronics all melted and the BTB was mixed to affect dissolution. The pans were taken from the oven and allowed to cool. All samples solidified.

One of the Pluronic samples (still in the weighing pan) was placed in the tank and a timer started. As the Pluronic, dissolved it released the BTB which was evident by a blue color developing. The rate of increase in the intensity of the blue was monitored by determining the % transmission at 617 nM using the spectrophotometer. A calibration curve was developed which allowed the calculation of the release rate.

The experiment was repeated for each of the subject Pluronics. To show the effect of this invention on the delivery pattern, BTB was dissolved in Pluronic F87 and was tested by the technique described above. The release pattern is clearly linear and this is supported by an R² of 0.9989. A plot of the derivative of this curve also supports the zero order hypothesis. Comparing this curve with that in Example 1, shows that the technique is effective in controlling the release rate. The Pluronic F87 appears capable of a uniform rate of release of 0.53 mg of BTB per minute. Diffusion from the polymer does not appear to be a contributing factor.

The other Pluronics were of higher molecular weight.

The release rates for the three polymers are summarized in the following table and graph.

[image]

### EXAMPLE 3

A measured amount of polyethylene glycol 1000 was incorporated into a 4″×4″×0.25″ square of a composite polymer matrix as taught in U.S. Pat. No. 6,617,014. Several sheets of paper were marked out in a square 12″ by 12″. Each square was weighed on an analytical balance to 0.1 mg. The foam square was wetted and the marked off portion of a piece of paper was scrubbed. Both the paper and the foam were died and weighed. The procedure was repeated with new sheets of paper. The increase in weight of each sheet is interpreted as the amount of PEG1000 delivered by scrubbing the paper. Roughly an equivalent amount of PEG1000 was delivered by each application.

### EXAMPLE 4

### Staining of a Hydrophilic Polyurethane

A hydrophilic polyurethane foam (HPUR) was produced by emulsifying equal portions of water and a hydrophilic polyurethane prepolymer (Hypol 2000, Dow Chemical USA). The foam was allowed to cure for 1 hour and then dried to constant weight. A 20 gram sample of the foam was placed in 1.0 liters of water containing 100 mg of bromothymol blue (BTB). The decrease in the intensity of the color was monitored with a spectrophotometer. FIG. 1 shows the spectral data. This indicates the extraction/immobilization of the BTB by the HPUR.

### EXAMPLE 5

Bromothymol Blue was immobilized in a sponge of hydrophilic polyurethane. The sponge was dried and then soaked in a 10% citric acid solution. The sponge was squeezed to remove excess solution and dried to constant weight.

The sponge was then immersed in water for 2 seconds. It was then removed and squeezed to remove excess water. The yellow color of the sponge indicated the present of citric acid in the sponge. The sponge was again immersed in fresh water, squeezed and inspected for color. This procedure was repeated 15 times, at which point the sponge was noticeably green in color, indicating the complete removal of the citric acid.

### EXAMPLE 6

A small amount of Hypol 2000 was emulsified with an equal portion of water. Before the emulsion solidified, it was brushed onto the side of a commercial sponge mop in a patch of about 1 inch by 2 inches. After curing, a solution of BTB was painted onto the HPUR patch and allowed to soak into it for 10 minutes. Excess dye was washed off with water. The sponge was then dried.

A portion of Pluronic F127 was melted and a small amount of citric acid was dissolved in it. While molten, it was painted over the HPUR patch with a significant amount of overlapping. Two grams of Pluronic were applied to the sponge in this manner.

A pail of water was prepared using a commercial floor washing soap.

Over a period of five days, the treated sponge mop was immersed in the pail, squeezed five times under the soap water, removed and squeezed dry. The color was inspected and the sponge left to dry in air for a minimum of four hours. After fourteen immersions, the color of the sponge changed from yellow to blue, indicating the end of its useful life.

What is claimed is:

1. A composition of matter adapted to release an active ingredient over a period of time at a linear rate comprising an active ingredient contained within a water-soluble or erodible polymeric matrix, said active ingredient/polymeric matrix mixture being dispersed within open cell or reticulated hydrophilic polyurethane.

2. A composition of matter adapted to release an active ingredient over a period of time at a linear rate comprising an active ingredient contained within a water-soluble or erodible polymeric matrix, said active ingredient/polymeric matrix mixture being dispersed within a composite comprising an open cell hydrophobic polyurethane foam, said hydrophobic foam having a plurality of surfaces defining a plurality of pores and having a coating of a substantially open cell hydrophilic polyurethane foam disposed upon said plurality of surfaces of the pores of said hydrophobic polyurethane foam.

3. A composition of matter as recited in claim 1 further containing an indicator molecule in said hydrophilic polyurethane.

4. A composition of matter as recited in claim 2 further containing an indicator molecule in the open cell polyurethane composite.
